(19) United States
(12) Patent Application Publication
(10) Pub. No.: US 2012/0191138 A1
(43) Pub. Date: Jul. 26, 2012

(54) USE OF FLEXIBLE BUSHINGS TO PROMOTE HEALING AND STABLE FRACTURE COMPRESSION IN ORTHOPEDIC TRAUMA PLATES

(76) Inventor: P. Douglas Kiester, Irvine, CA (US)

(21) Appl. No.: 13/011,598

(22) Filed: Jan. 21, 2011

(51) Int. Cl. A61B 17/88; A61B 17/80

(52) U.S. Cl. 606/281; 606/289

(57) ABSTRACT

In one embodiment, an orthopedic plate system is provided, comprising one or more orthopedic plates, each having one or more openings, a plurality of screws for securing one or more bones to said plates through said openings, and, means for flexibly locking said screws to said plates so that controlled motion of said bones is enabled after the securing of said bones to said plates. One advantage of said system is that of enhancing the healing of said bones and/or their fractures. A screw and bushing assembly, which flexibly locks said screw to an orthopedic plate, and a method of installing said orthopedic plate, are also disclosed.

### BACKGROUND OF THE INVENTION

1. Field of the Invention

The present invention relates generally to the field of orthopedic surgery and particularly to an orthopedic plate and screw system using bushings.

2. Description of the Related Art

How to best promote rapid and robust healing of fractured bone without residual deformity continues to be the primary goal of orthopedic trauma surgeons. Plate and screw constructs have long been and continue to be a staple in the surgeon's toolbox. Classically, these plates work by a tightened screw pulling and holding the plate tightly against the bone. If the screw loosens, the construct will generally fail resulting in either a healed, deformed bone and/or a non-healed, unstable bone.

One of the more recent advances in the standard plate and screw system was the advent of “locking” screws. These screws were designed for the screw head to become rigidly fixed to the plate. This improved pull-out strength, and allowed complex, fragmented fractures to be held firmly in place. An added bonus is that the locking screws don't come loose from the plate.

The primary problem with locking plates is that these constructs are so rigid the fractures take much longer to heal. The same effect is observed when external fixators are used. If there is no mechanical load allowed across the fracture site, the chemical, electrical, and mechanical signals generated by loading the bone aren't generated. The result is greatly delayed, or, in some cases, the fracture healing is completely arrested. The key to healing is to put compression across the fracture site. Classically, this has been done by placing the screws in the bone in such a way that once tightened the plate acts like a tension band while the screws push against the plate to force the fractured ends together at high pressure. While this does get the bone to heal more rapidly, local bone necrosis at the fracture site has long been recognized. Allowing for intermittent, controlled loading/compression without loss of fracture reduction would be the ideal.

A lesser problem with current locking plates is that once the screw head comes in contact with the plate, the screw locks to the plate. There is no further tightening possible. The plate remains suspended above the bone. Because of this mechanical similarity with external fixators which also do not come in contact with the bone, these locking plates have been promoted as “internal external fixators”. If the surgeon wants the plate to be in firm contact with the bone, he must use specially designed screws to pull the plate to the bone, then put in the locking screws.

A third problem with the current locking plates is cold welding of the screw head to the plate which can make screw removal surgically challenging.

The problems and the associated solutions presented in this section could be or could have been pursued, but they are not necessarily approaches that have been previously conceived or pursued. Therefore, unless otherwise indicated, it should not be assumed that any of the approaches presented in this section qualify as prior art merely by virtue of their presence in this section of the application.

### BRIEF SUMMARY OF THE INVENTION

In one embodiment, a specially designed bushing, or similar means, is used between one or more screws and an orthopedic plate, for locking the plate to a bone in a non-rigid, micro-motion-enabling and controlling manner. The direction and amount of flexibility that the screw is allowed is carefully engineered. Unlike any of the prior art, the screw is held to the bushing by friction (not screw threading) between the bushing and the screw. Because the bushing holding the screw is flexible, the screw has some flexibility. Again, the direction and amount of flexibility that the screw is allowed is carefully engineered. Some adjustments to that flexibility can be made by choices allowed to the surgeon in the operating room.

A flexible locking is achieved in the sense that friction and flexibility relieving stress on the bushing is not allowing the screw to back out of the plate. When the screw does “flex,” it springs back to its original position. Thus, an advantage is the controlled micro-motion, which enhances the healing of said bone and/or its fracture. Another advantage is allowing the plate to be firmly pulled onto the bone if the surgeon so chooses. Yet, another advantage is that when (and if) the screw needs to be removed, there is no cold weld, so the screw head should not strip on removal as is fairly common with the current locking plates.

In the prior art, when the screw is allowed to move it stays moved in its new position. The proposed bushing is flexible. Thus the screw has a tendency to spring back to its original position after loading. Not only is the fracture loaded with patient movement in the first place, but the loading is relieved when the patient rests. This means that the compression of the fracture will be intermittent, which is a much more beneficial physiologic situation for healing than the current technologies. Because only specifically controlled micromotion is allowed, there is no loss of fracture reduction.

The friction fit between the bushing and the screw allows the surgeon to stop tightening the screw at any degree of pressure desired between the plate and the bone. The friction between the bushing “locks” the screw, preventing it from backing out at any point the surgeon quits tightening the screw. Cold weld between the bushing and screw, with the resultant stripping of screw head, should not occur, making removal of the hardware surgically more predictable.

Because it is still a locking plate, the advantages for dealing with complex fractures are still realized.

In another embodiment, the screws may, at a pressure level set during installation, be allowed to slide after the installation, and thus, allowing the fracture to be compressed while still controlling its alignment and rotation. After any sliding there will still be controlled intermittent loading to promote healing. The post-installation controlled compression of the fracture is also very advantageous to healing.

In another embodiment, the micro-motion enabling bushing may be shaped to have the screw inserted into the plate in a pre-engineered fixed angle, or so that the screw may be inserted at a variable angle.

The above embodiments and advantages as well as other embodiments and advantages will become apparent from the ensuing description and accompanying drawings.

### BRIEF DESCRIPTION OF THE DRAWINGS

For exemplification purposes, and not for limitation purposes, embodiments of the invention are illustrated in the figures of the accompanying drawings, in which:

FIG. 1a illustrates a perspective view of a portion of an orthopedic plate system according to one embodiment.

FIG. 1b illustrates a longitudinal-sectional view of the orthopedic plate system from FIG. 1a.

FIG. 2 illustrates a cross-sectional view of an orthopedic plate system as in FIG. 1a, taken through one of the screws, after installation.

FIG. 3a illustrates a perspective view of a portion of an orthopedic plate system according to another embodiment.

FIG. 3b illustrates a top view of an orthopedic plate system as in FIG. 3a.

FIG. 3c illustrates a partial longitudinal-sectional view of an orthopedic plate system as in FIG. 3a, after installation.

FIG. 3d illustrates a perspective view of an orthopedic plate system as in FIG. 3c.

FIG. 4a illustrates a top view of a portion of an orthopedic plate system according to another embodiment.

FIG. 4b illustrates a perspective view of an orthopedic plate system as in FIG. 4a.

FIG. 4c illustrates a partial longitudinal-sectional view of an orthopedic plate system as in FIG. 4b.

FIG. 5a illustrates a top view of a portion of an orthopedic plate system according to another embodiment.

FIG. 5b illustrates a perspective view of an orthopedic plate system as in FIG. 5a.

FIG. 5c illustrates a longitudinal-sectional view of an orthopedic plate system as in FIG. 5b.

FIG. 6a illustrates a perspective view of a portion of an orthopedic plate system according to another embodiment.

FIG. 6b illustrates a perspective view of a portion of an orthopedic plate system according to another embodiment.

FIG. 6c illustrates a cross-sectional view taken along lines 6c-6c of FIG. 6b.

FIG. 6d illustrates a cross-sectional view taken along lines 6d-6d of FIG. 6b.

FIG. 6e illustrates a partial longitudinal-sectional view of an orthopedic plate system as in FIG. 6b.

FIG. 7a illustrates a perspective view of a portion of an orthopedic plate system according to another embodiment.

FIG. 7b illustrates a cross-sectional view of an orthopedic plate system as in FIG. 7a, taken through one of the screws.

FIGS. 8a-c illustrate perspective views of an orthopedic plate system according to one embodiment being positioned at various distances from a fractured bone.

FIG. 9 illustrates a perspective view of an orthopedic plate system according to another embodiment.

FIG. 10a illustrates a perspective view of a portion of an orthopedic plate system according to another embodiment.

FIG. 10b illustrates a cross-sectional view taken along lines 10b-10b of FIG. 10a.

FIG. 11a illustrates a partial top view of a plate-bushings subsystem, wherein the holes 1113 in the bushings have different diameters, and a side view of the corresponding screws according to several embodiments.

FIG. 11b illustrates partial cross-sectional views taken through the holes 1113 in FIG. 11a, after the screws were installed.

FIG. 12 illustrates the side view of a screw having a tapered neck according to another embodiment.

FIGS. 13a-b illustrate a perspective and a side view, respectively, of an orthopedic plate system, using a “bushing” system comprising two flexible members 1301, to allow micromotion along one axis only, in accordance to another embodiment.

### DESCRIPTION OF THE PREFERRED EMBODIMENTS

What follows is a detailed description of the preferred embodiments of the invention in which the invention may be practiced. Reference will be made to the attached drawings, and the information included in the drawings is part of this detailed description. The specific preferred embodiments of the invention, which will be described herein, are presented for exemplification purposes, and not for limitation purposes. It should be understood that structural and/or logical modifications could be made by someone of ordinary skills in the art without departing from the scope of the present invention. Therefore, the scope of the present invention is defined only by the accompanying claims and their equivalents.

The terms micro-motion and motion are used herein interchangeably.

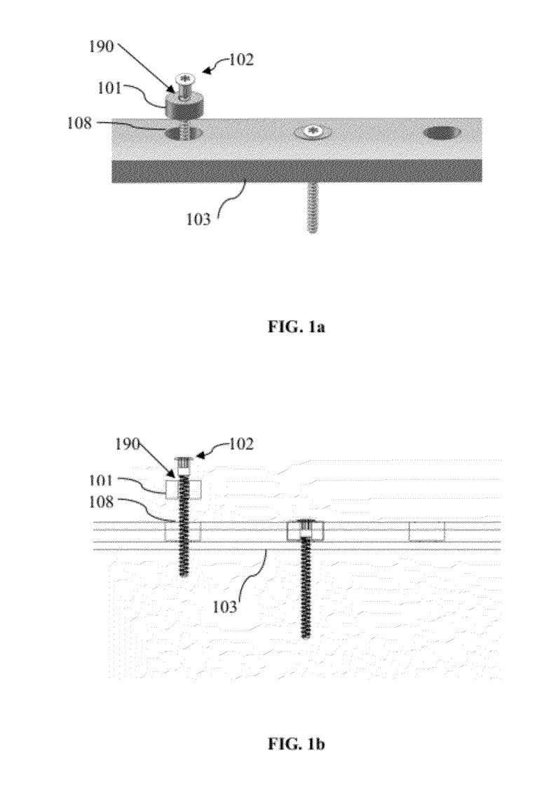

FIG. 1a and FIG. 1b illustrate, respectively, a perspective view and a longitudinal-sectional view of a portion of an orthopedic plate system according to one embodiment. The plate 103 has openings 108, which are sized to accommodate bushing 101. A bone screw 102 is used to be passed through the hole 190 of the bushing 101 in order to secure a bone to plate 103. Initially, before the tightening of the screw 102, the bushing 101 may engage movably the plate opening 108 and the screw 102. After the tightening of the screw 102, the bushing 101 engages the screw 102 and the plate opening 108 to form a flexible, motion enabling, friction lock. Hence, after the installation of the screws 102, a flexible friction lock is formed among the plate 103, the bushings 101 and the screws 102. The friction lock has the advantage of being able to allow the screw to be tightened to any degree desired.

The flexible friction lock, as opposed to a rigid friction lock is beneficial to the healing of the bone. The amount, the type and/or direction of the motion the bone or bones will be allowed to experience after the installation of the orthopedic plate system can be pre-determined as will be described in detail below. Thus, this orthopedic plate system gives the surgeon the ability to control what type and how much motion the bone(s) will experience after the surgery according to the specifics of the surgical intervention (i.e., type of fracture, type of bones being secured, etc). The flexible friction lock allows the screw to be fixed in a non-rigid fashion. This promotes carefully controlled and limited micromotion which is beneficial to the healing of the bone.

The flexible friction lock is the result of the screw 102 pressing onto the bushing 101, which is made of an elastic material, and which, in turn, presses onto the surface of the opening 108. The material used for the bushing may be any material acceptable for medical use and having desired elastic properties, such as rubber, silicon rubber, high density polypropylene or others.

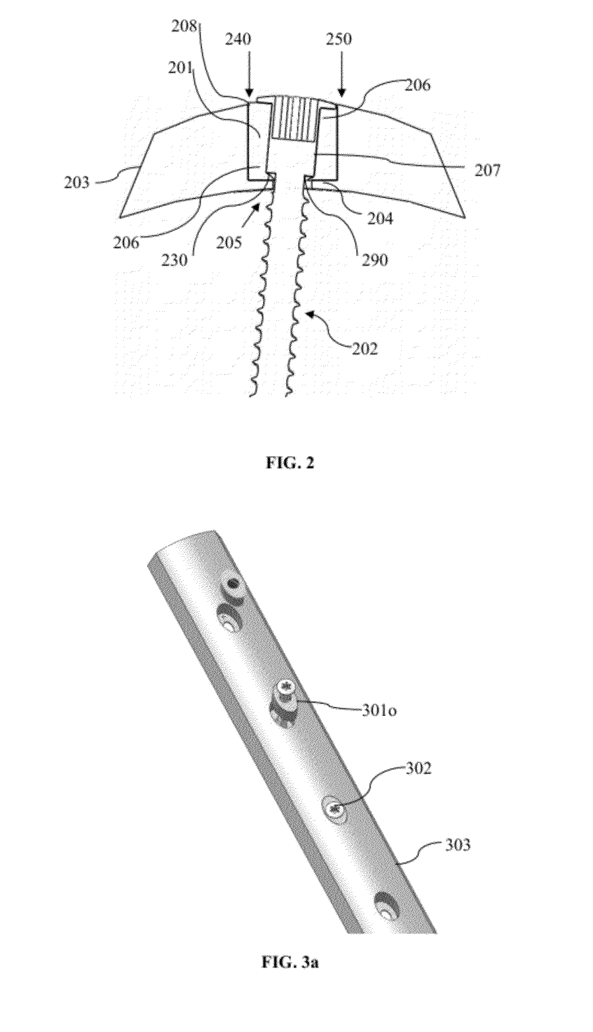

FIG. 2 illustrates a cross-sectional view of an orthopedic plate system as in FIG. 1a, taken through one of the screws, after installation. As explained before, after the installation of the screw 202 into a bone, a flexible friction lock is created among the plate 203, the bushing 201 and the screw 202. Again, the flexible friction lock is the result of the screw 202, and more precisely its neck 207, pressing onto the surface of the hole 290 of bushing 201, which is made of an elastic material, and, in turn, the bushing 201 pressing onto the surface of opening 208 of plate 203 along the 240 and 250 lines. The screw neck 207 does not have threads. It can be finished rough or smooth to grip the bushing to the desired degree. In order to be effective in exercising pressure onto the bushing 201, the screw neck 207 has a diameter which is preferably larger than the diameter of the bushing hole 290. As illustrated in FIG. 2, for easing the progression of the screw neck 207 into the bushing hole 290, the leading end 230 of the screw neck 207 may be tapered.

One way of controlling how much pressure the screw neck 207 exercises onto the bushing 201, and therefore, how much post-installation motion is allowed, is by pre-determining the diameter of the screw neck 207. For example, when all other factors are equal (e.g., shape and size of the bushing, density and elastic properties of the bushing material, diameter of the bushing hole 290, etc), a greater diameter of the screw neck 207 translates into lower flexibility of the friction lock, and therefore, into less post-installation bone motion allowed. This is because less room remains between the screw neck 207 and the plate 203, and because, all else constant, the more compressed the bushing 201 is, the less elastic it becomes. Hence, for a given bushing 201, should the surgeon determine in a particular case that less post-surgery motion should be experienced by the bone(s) in order to achieve the best healing results, he/she may choose a screw 202 with a neck 207 having a correspondingly greater diameter.

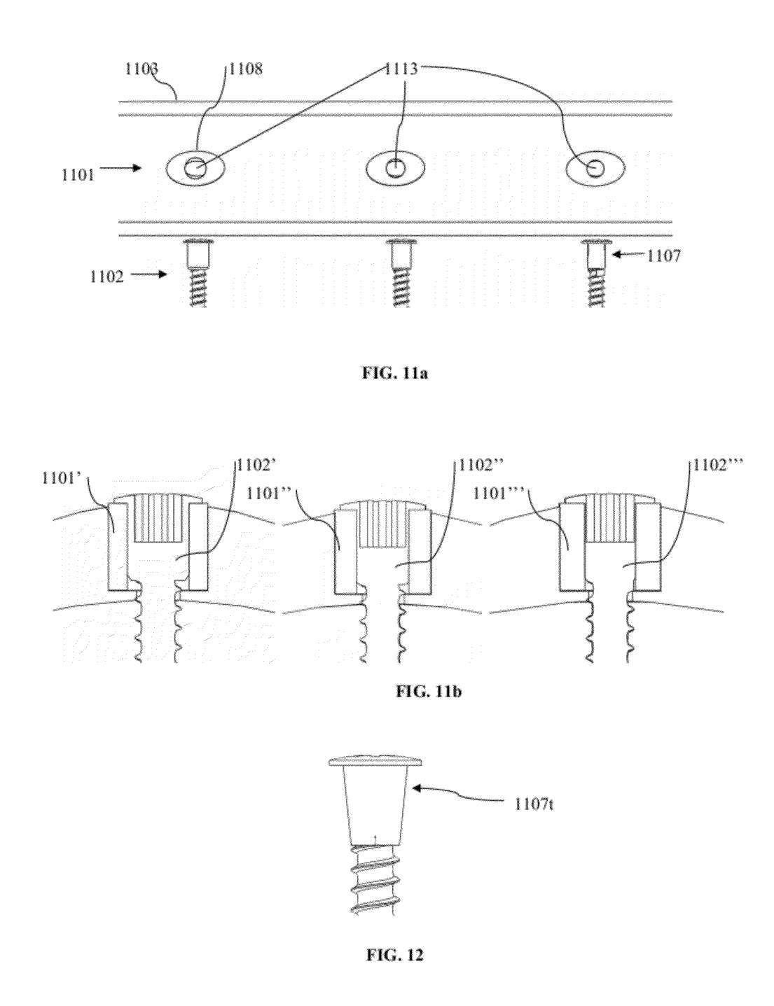

There are obviously many other ways to control the amount of motion the bone(s) will be allowed to experience after surgery. For example, all else constant, the greater the elasticity of the material used for the bushing, the greater the amount of motion allowed. As another example, the greater the thickness of the bushing wall, meaning the distance between the interior surface of the bushing 201, created by the bushing hole 290, and the exterior-opposite surface of the bushing 201, the greater the amount of motion allowed is. As another example, all else constant, the amount of motion allowed can be controlled by varying the degree of tightening of a screw 202 having a tapered neck 1107t as depicted in FIG. 12. The tighter the screw, the less the amount of motion allowed is.

Furthermore, the maximum amount of motion the screw 202, and consequently the bone, is allowed may be firmly controlled by pre-determining the area of the exit end of opening 208. As illustrated in FIG. 2, the diameter of the opening 208 is may be smaller at its exit end 205, thus blocking the screw 202 when, due to movement, the screw 202 reaches the upper limit of a pre-determined range of motion (e.g., one to five degrees).

In addition, for example when the bushing 201 has a cylindrical shape, and therefore, naturally allowing equal range of motion radially, the shape of the exit end 205 of opening 208 may be so predetermined and sized as to allow a range of motion that is greater in one plane, and smaller in another, for example, a perpendicular plane. Such a shape may be an oval, oblong or obround shape. Opting for a smaller exit end 205 has a secondary benefit. It creates the shelf 204 around the exit end 205, thus preventing the bushing-screw assembly from pulling out of the plate 203.

In FIG. 2, a five degree motion is depicted. Ranges of motion of the screw 202 included in the zero to five degrees interval (e.g., 0-3 degrees) may be preferred in the transverse planes of the plate 203, to enhance healing while still controlling, for example, rotational movement of the bone. However, other ranges may be used.

As depicted in FIG. 2, when the screw 202 moves from its initial “straight” position, diagonally opposite sections 206 of the bushing 201 are compressed. The strength of this orthopedic plate system, and that of the other embodiments described below, characterized by the flexible friction lock, is that after the deformation force is removed, the screw 202 tends to return to its initial position. This puts the fracture under intermittent motion (e.g., compression), thus enhancing its healing.

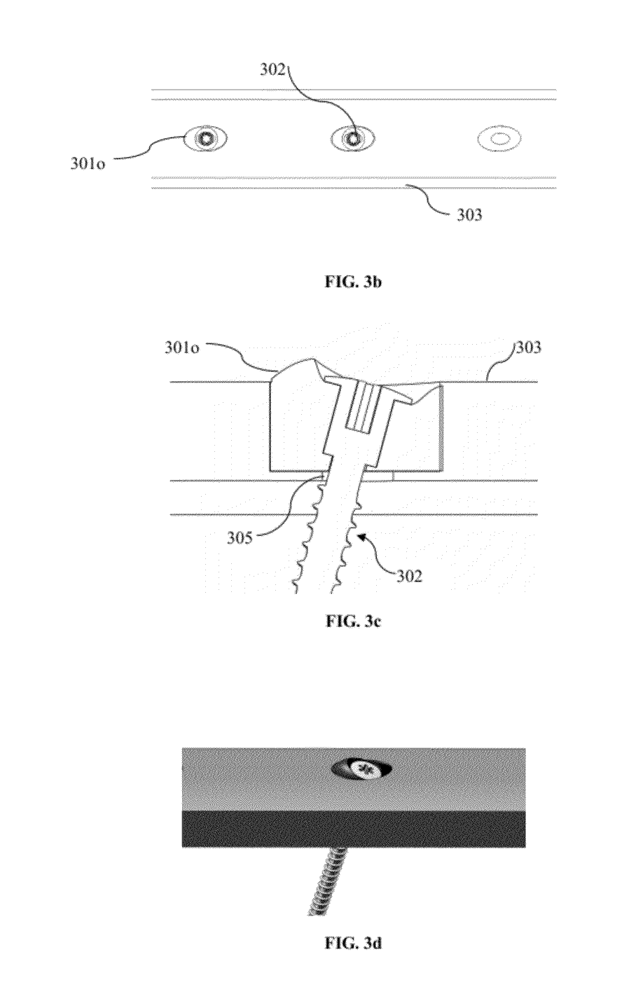

FIG. 3a and FIG. 3b illustrate, respectively, a perspective view and a top view of a portion of an orthopedic plate system according to another embodiment. In this embodiment, the bushing 301o has an oval shape. The oval shape of the bushing 301o may be used to control not only the amount of motion allowed as described above, but also the direction and/or plane in which more or less motion, the screw 302 is allowed to undergo after installation. Consequently, the amount and the type of motion the bone(s) experience after surgery is controlled. For example, where, as illustrated in FIGS. 3a-b, the oval bushing 301o is installed with its longer axis in a parallel position with the longer axis of plate 303, the screw 302 is allowed to undergo, after installation, more motion in the longitudinal plane(s) and less motion in the transverse plane(s). This means that the bone(s) secured with this plate system is/are allowed to experience more, for example, compression, and less, for example, rotation motion, after the surgery.

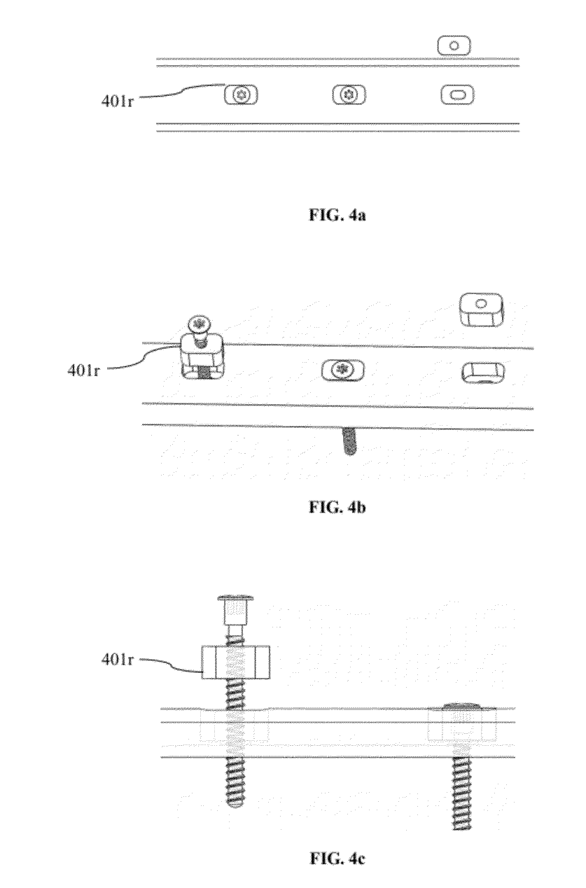

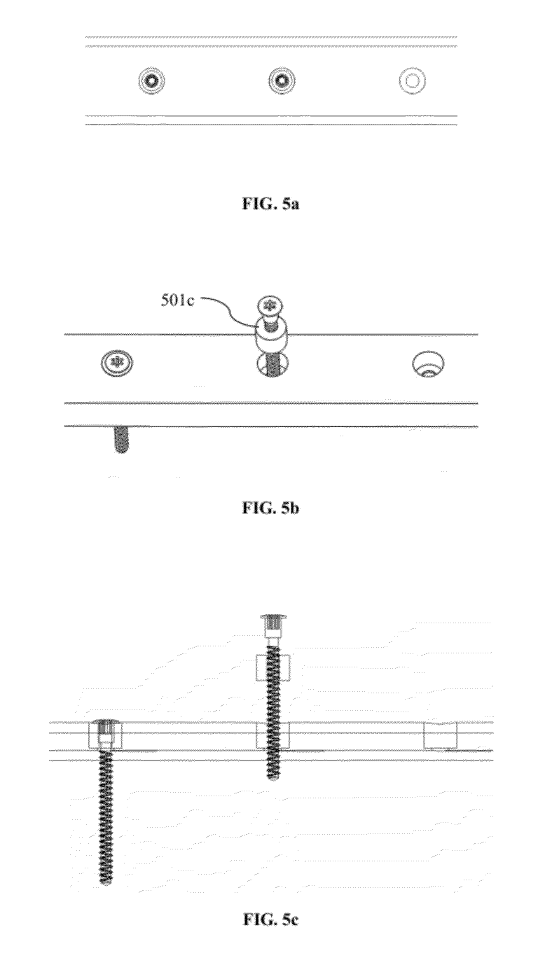

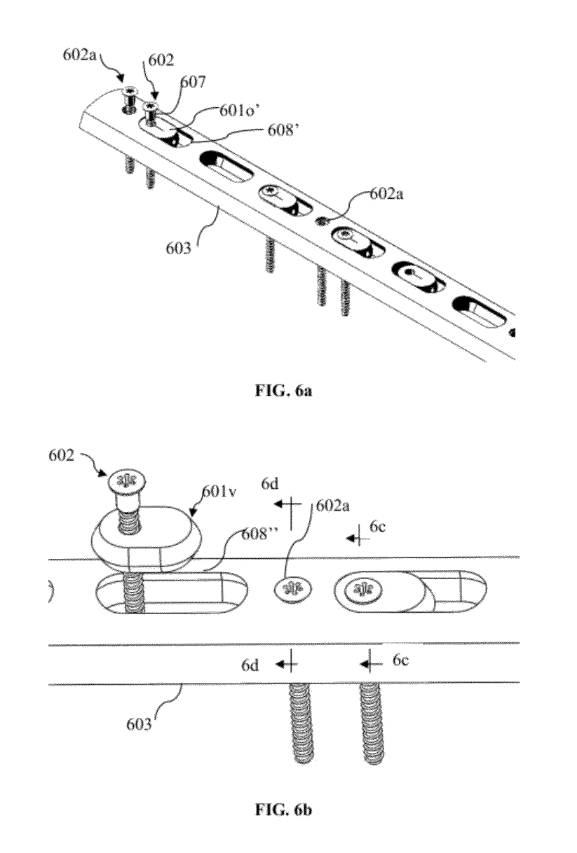

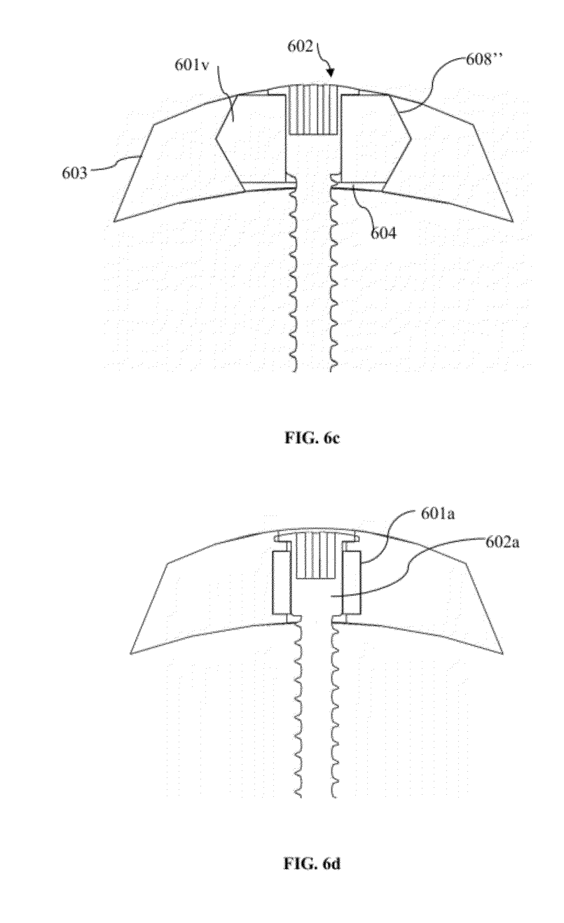

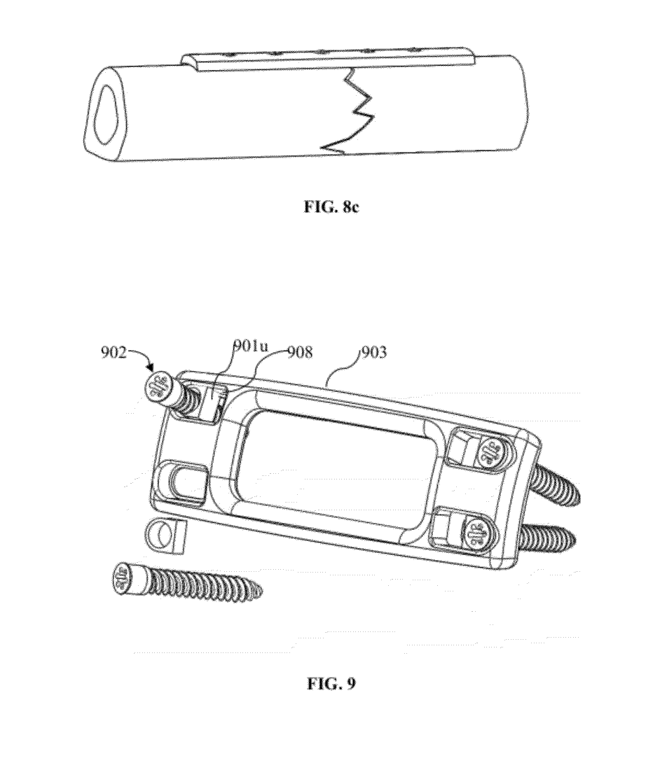

A similar bone motion controlling effect may be achieved using other oblong bushing such as the rectangular bushing 401r in FIGS. 4a-c, obround bushing 601o′ in FIG. 6a or v-shape bushing 601v in FIGS. 6b-c. This is in contrast with the bushings which permit equal amount of motion in all radial directions or planes, such as the cylindrical bushings 101 in FIGS. 1a-b, 201 in FIG. 2, 501c in FIGS. 5b and 1001c′ in FIG. 10b, or, the spherical bushing 701s in FIGS. 7a-b. For such bushings, other means for controlling the maximum amount of motion allowed in a certain direction or plane may be employed, such as by pre-determining the shape and size of the exit end 205 of the plate opening 208, as discussed earlier under the FIG. 2 description.

FIG. 3c illustrates a partial longitudinal-sectional view of an orthopedic plate system as in FIG. 3a, after installation. The description of this embodiment as to how the flexible friction lock is achieved, and how the amount and type of post-surgery motion is controlled, is similar to what was described earlier under FIGS. 1a-b and FIG. 2. The difference is that in this embodiment an oval bushing 301o is employed. Particularly, the oval bushing 301o is installed with its longer axis in a parallel position with the longer axis of plate 303. Thus, the screw 302 is allowed to undergo, after installation, more motion in the longitudinal plane(s) and less motion in the transverse plane(s). This means that the bone(s) secured with this plate system is/are allowed to experience more, for example, compression, and less, for example, rotation motion, after the surgery.

Fifteen degrees range of allowed motion is illustrated in FIG. 3c (see blockage exercised by the exit end 305). Ranges of motion of the screw 302 included in the zero to fifteen degrees interval (e.g., 0-12 degrees) may be preferred in the longitudinal planes of the plate 303, to enhance healing while still controlling, for example, compression movement of the bone. However, other ranges may be used.

FIG. 6a illustrates a perspective view of a portion of an orthopedic plate system according to another embodiment. In this embodiment, the assembly created by the bushing 601o′ and the screw 602 is allowed to slide after installation a predetermined distance at a predetermined pressure and in a predetermined direction (longitudinally in FIG. 6a; however, other directions may be used (e.g., transversal direction)). The maximum sliding distance may be predetermined mainly by pre-sizing the difference between the lengths of bushing 601o′ and sliding opening 608′ (or the widths if transversal sliding is desired).

The sliding direction may be predetermined by selecting the location of the size difference between bushing 601o′ and opening 608′ (i.e., lengthwise as in FIG. 6a, widthwise, or otherwise). The pressure at which sliding will occur may be controlled by predetermining the values of the factors discussed earlier under FIG. 2 (e.g., diameter of the screw neck 607, shape and size of the bushing 601o′, density and elastic properties of the bushing material, etc), or other factors, such as the friction properties of the materials used for the bushing 601o′ and plate 603. For example, all else constant, if the surgeon chooses a screw 602 which has a greater diameter at its neck 607 section, a stronger flexible friction lock is achieved, and consequently, a greater pressure will be required to cause sliding.

Also, as suggested in FIG. 6a, in order to decrease the level of pressure at which the sliding will occur, the surgeon may choose to leave some of the openings 608′ unused so that the total strength of the flexible friction locks is decreased. In other words, the surgeon has the option to control the pressure at which, the fracture and/or the bone(s) will be allowed to experience, for example, contraction motion. This is important as the desired sliding pressure may be different from fracture to fracture.

And, as explained above, the surgeon may also control the direction and the maximum amount of sliding by choosing the appropriate plate design (e.g., as the one in FIG. 6a or, for example, a plate wherein the openings 608′ are positioned transversally). Furthermore, in order to give more options to the surgeon, the plate 603 may have sliding openings 608′ of various orientations, shapes and/or sizes, and corresponding bushing-screw subassemblies may be provided, for the surgeon to use the ones that are most desirable for enhancing the healing of the subject fracture and/or bone(s).

It should be apparent from FIG. 6a, that the entire lateral-exterior surface of bushing 601o′ and the corresponding surface of opening 608′ are round. The round surfaces allow for an engagement between the bushing 601o′ and opening 608′ which prevent, after installation, both, the pulling and the pushing of the bushing-screw assembly out of the plate 603. It may be sufficient to have only two opposite sections (the longitudinal sections in this example) of the lateral-exterior surface of the bushing 601o′ (and the corresponding sections of the opening 608′) as round surfaces in order to achieve a similar, although likely less desirable, result. It should be noted that the round surface configuration offers also some flexibility in choosing the angle at which the screw 607 is initially inserted into the bone.

It should also be apparent that one or more static screws 602a may initially be used. After the surgery, a few weeks (or months) into the healing, the static screw(s) 602a may be removed and the plate becomes dynamic. Instead of immediately compressing after static screw removal, the fracture and/or the bone will only compress to the degree that the forces exceed the pre-determined force/pressure necessary to slide the bushing(s) 601o′ along the sliding opening 608′ in the plate 603. Once compressed, the fracture is again exposed to intermittent compression as the flexible screw/bushing construct is exposed to force, and then the screw 602 springs back when the pressure is removed. This intermittent stress has been shown to enhance bone healing.

FIG. 6b illustrates a perspective view of a portion of an orthopedic plate system according to another embodiment. This embodiment is very similar with the one described above and depicted in FIG. 6a. Thus, the above description pertains to this embodiment in large part. The difference here is that the lateral-exterior surface of bushing 601v and the corresponding surface of opening 608″ are v-shaped, which is another effective way of preventing the assembly formed by screw 602 and bushing 601v from pulling or pushing out of the opening 608″ of plate 603. Again, one or more static screws 602a may be initially used to firmly hold the fracture and/or bone(s) in place and later taken out of the bone in order to allow controlled motion (e.g., compression), and thus, enhance healing.

FIG. 6c illustrates a cross-sectional view taken along lines 6c-6c of FIG. 6b. Again, the v-shaped lateral-exterior surface of bushing 601v and the corresponding surface of opening 608″ is another effective way of preventing the assembly formed by screw 602 and bushing 601v from pulling or pushing out of the plate 603. In addition, a shelf 604, similar with the shelf 204 described under FIG. 2, may improve even more and practically eliminate the risk that the assembly formed by screw 602 and bushing 601v will pull out of the plate 603 after installation.

FIG. 6d illustrates a cross-sectional view taken along lines 6d-6d of FIG. 6b. As suggested here, the static screw 602a may have its own bushing 601a, which, however, has relatively thinner walls. A surgeon may choose to use such a “static” screw, which has some limited degree of mobility, if the surgeon determines that even in the beginning, the particular fracture and/or bone(s) would benefit from such limited mobility offered by this flexible friction lock as opposed to a rigid lock to which the fracture/bone(s) would be subjected by a true static screw with no bushing at all.

FIG. 6e illustrates a partial longitudinal-sectional view of an orthopedic plate system as in FIG. 6b. Specifically, it depicts a “static” screw 602a having its own bushing 601a, a dynamic screw 602 surrounded by a v-shape bushing and plate 603. Again, the surgeon may choose to use initially one or more true static screws (no bushing at all), or less “static” screws 602a having a relatively small bushing 601a, in conjunction with the dynamic screws 602 having bushing 601v for example.

The surgeon may also skip the use of static crews, whether without or with bushing, and use only dynamic screws 602 and bushings 601v. Hence, depending on the particular fracture and/or bones, the surgeon may plan for a one-phase, two-phase, three-phase, etc, process for securing the fracture/bone(s) and promoting healing. For example, the surgeon could start with true static screws (no bushing at all) and dynamic screws, continue in phase two by replacing the true static screws with less “static” screws (having a small bushing) and ending with phase three by removing the less “static” screws so that the fracture/bone(s) may experience increased degree of motion as predetermined to be permitted by the dynamic screws. Thus, in this example, the fracture and/or the bone(s) will experience a progressive degree of motion: from zero (phase one), to some degree of motion (phase two), and finally, to an increased, yet controlled degree of motion (phase three).

It should be understood that the direction and degree of flexibility for the less “static” screws 602a (having a bushing 601a) may be also predetermined, the same way as for the dynamic screw/bushing assembly 602/601v, as described earlier under FIGS. 1a-b and FIG. 2. Hence, the surgeon may be given the option to choose, for a particular fracture/bone(s), true static screws (no bushing) or less “static” screws (with bushing) configured to allow various degrees of initial motion.

FIGS. 7a and 7b illustrate, respectively, a perspective and a cross-sectional view of a portion of an orthopedic plate system according to another embodiment. The advantage of this embodiment is that, besides achieving flexible/dynamic friction lock, the surgeon has a wide range of options as to what angle to insert screws 702 at into the bone. This is facilitated by the spherical bushings 701s.

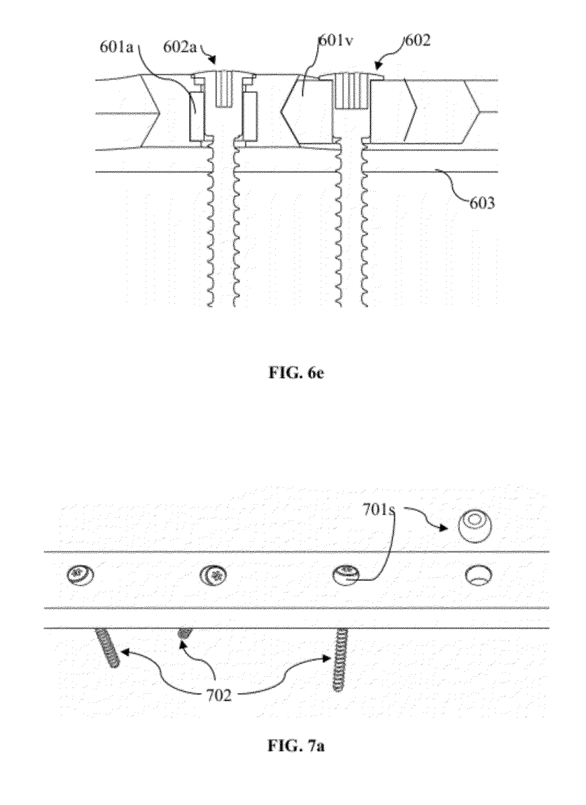

FIGS. 8a-c illustrate perspective views of an orthopedic plate system 800 according to one embodiment being positioned at various distances from a fractured bone 810. The advantage of the flexible friction lock plate is that the surgeon may choose to lock the plate at any of the positions depicted in the three figures or at any other intermediate positions. The position depicted in FIG. 8a is the typical position with a standard locking plate. On a standard locking plate, once the heads of the screws contact the plate they lock, and the plate's position becomes fixed in space. Some surgeons feel that this position gives a mechanical advantage. Some surgeons like the position depicted in FIG. 8b because it allows blood to circulate under the plate, which, they feel, promotes healing. The tight against the bone position depicted in FIG. 8c is standard for many surgeons. Again, with the flexible friction lock, the surgeon has the choice to lock the plate where he wants, or finds appropriate for the particular case, and still preserve the controlled motion features, which again, enhance the healing.

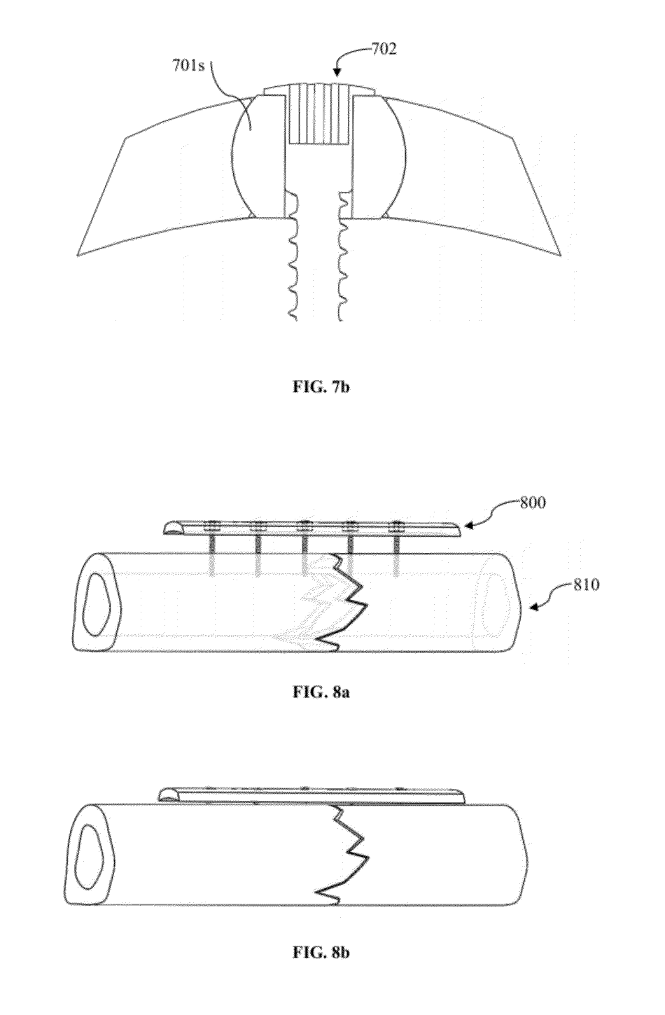

FIG. 9 illustrates a perspective view of an orthopedic plate system according to another embodiment. The description here is similar with the descriptions for the other sliding plates (see descriptions of FIGS. 6a-b above). This cervical spine fusion plate is configured to enhance the fusion of a disk space after surgery by flexibly locking the plate 903 and allowing the assemblies formed by screws 902 and bushings 901u to slide inside openings 908 at a predetermined pressure and in a predetermined direction.

FIG. 10a illustrates a perspective view of a portion of an orthopedic plate system according to another embodiment. It may be desirable to preload the bushing on the plate. It can be mounted with adhesive and/or mechanically. An optional mechanical mounting of a sample cylindrical bushing is depicted here, which is better understood by turning to FIG. 10b (see description below).

FIG. 10b illustrates a cross-sectional view taken along lines 10b-10b of FIG. 10a. The bushing 1001c′ may be preloaded into the opening 1008 of plate 1003. The bushing 1001c′ is constrained to remain there by the shelf 1004 and shoulder 1012.

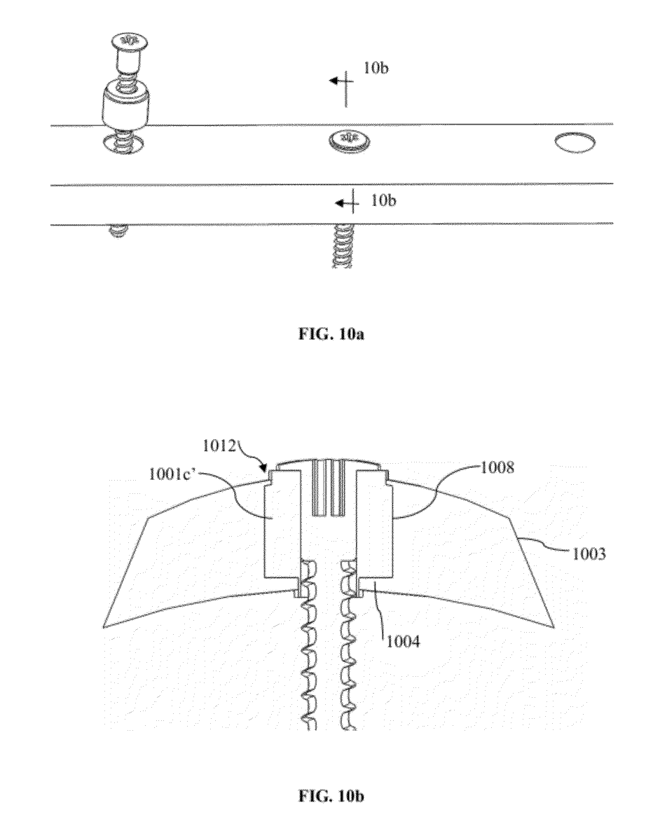

FIG. 11a illustrates a partial top view of a plate-bushings subsystem formed by the plate 1103 and bushings 1101, wherein the holes 1113 in the bushings 1101 have different diameters, and a side view of the corresponding screws 1102 having different diameters at their neck section 1107, according to several embodiments. For exemplification purposes, the three bushings are oval in shape, made of the same material and of equal exterior size. However, starting from the left, their holes 1113 are increasingly smaller in diameter. This means that after the installation of the corresponding screws 1102 (as seen in FIG. 11b), the lowest degree of motion will be allowed by the first (starting from the left) bushing-screw assembly (formed by the bushing 1101′ and screw 1102′ (see FIG. 11b)). This is because, as explained in detail earlier in this description, the walls of the first bushing (1101′ in FIG. 11b) is the narrowest among the three, and therefore, there is the least elastic material to be compressed.

By analogy, a higher degree of motion will be allowed by the assembly formed by the bushing 1101″ and the screw 1102″ and the highest degree of motion will be allowed by the assembly formed by the bushing 1101′ and the screw 1102′. Thus, for the same size and shape of opening 1108, the surgeon may choose screw-bushing assemblies which allow the most desirable degree of motion in order to enhance the healing of the particular fracture and/or bone(s).

Many other configuration possibilities exist, which may afford the surgeon the option of choosing a screw-bushing assembly which will allow the desired amount and direction of motion for the particular case. For example, bushings of the same shape and size may be made available to the surgeon, which are made of different elastic materials or of the same elastic material but of different densities, and therefore, each bushing being capable of enabling a different degree of motion. For easy use, the bushings may be labeled to indicate the degree of motion allowed (e.g., one degree, two degrees, etc). In another example, a plurality of screws having various diameters at their neck section, may accompany a particular bushing. Thus, as described before under FIG. 2, the surgeon may choose a screw of greater diameter if he/she determines that less post-surgery motion should be experienced by the particular fracture and/or bone(s). The screws and/or the bushing-screw assemblies may also be labeled.

FIG. 12 illustrates the side view of a screw having a tapered neck 1107t according to another embodiment. Again, the tapered neck 1107t may help correlate the strength of the flexible/dynamic friction lock with the degree of screw tightening during installation.

It is to be understood that the various bushing configurations described in this description, may be pre-mounted on the plate, on the screw, or, it may be used as a washer that's added at the time of insertion.

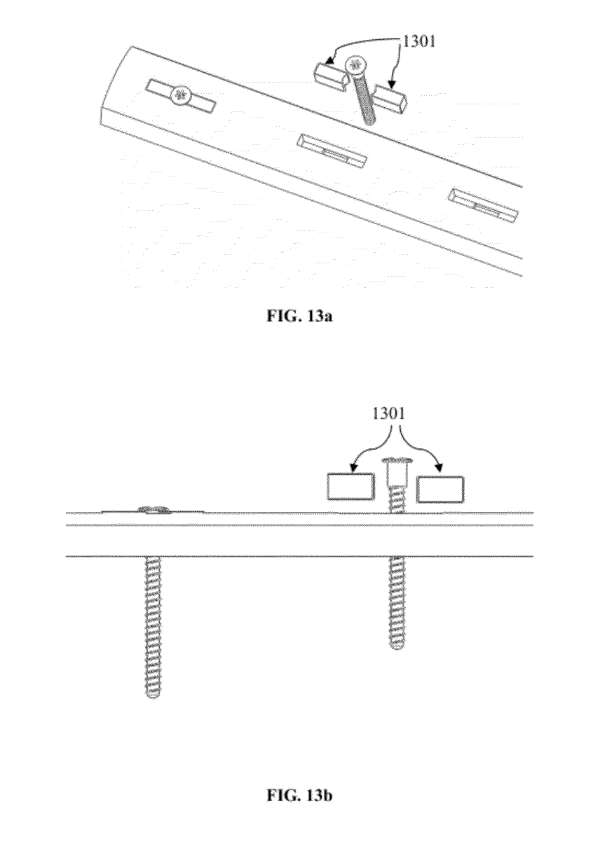

As illustrated in FIGS. 13a and 13b, other configurations and/or means may be used to allow controlled post-surgery motion of the screw and consequently of the bone. For example, halves of bushings, such as the ones depicted in FIGS. 13a and 13b (1301), or quarters of bushings, or equivalents, may be used alone or in combination to allow motion only in certain direction(s) or plane(s), i.e. where the section(s) is/are used, and prevent motion in other direction(s) or plane(s), i.e. where the section(s) is/are not used.

Other flexible/elastic structures may be used between the screw and the plate, as long as they are suited for achieving controlled post-surgery motion of the screws, and therefore, of the bone to enhance healing. For example, spring-based structures may be used. Such a structure may comprise, for example, an interior cylinder with an opening for the passage of the screw, an exterior cylinder larger in diameter than the interior cylinder and configured to sit in the plate opening, and a plurality of springs positioned between the two cylinders in, for example, a perpendicular (to both cylinders) arrangement. The interior cylinder and the exterior cylinder may need to have a side cut (or two side cuts, hence, converting in half-cylinders) so that they expand during the insertion of the screw. Such a structure would the functional equivalent of a cylindrical bushing described earlier in this description. Similarly, the functional equivalent of an oval, obround, spherical, etc bushing may be constructed.

Although specific embodiments have been illustrated and described herein for the purpose of disclosing the preferred embodiments, someone of ordinary skills in the art will easily detect alternate embodiments and/or equivalent variations, which may be capable of achieving the same results, and which may be substituted for the specific embodiments illustrated and described herein without departing from the scope of the present invention. Therefore, the scope of this application is intended to cover alternate embodiments and/or equivalent variations of the specific embodiments illustrated and/or described herein. Hence, the scope of the present invention is defined only by the accompanying claims and their equivalents.

What is claimed is:

1. An orthopedic plate system comprising:
one or more orthopedic plates, each having one or more openings;
a plurality of screws for securing one or more bones to the plate through said openings; and,
means for flexibly locking said screws to the plate so that controlled motion of the bone is enabled after the securing of the bone to the plate.

2. The orthopedic plate system according to claim 1, wherein at least one of said openings is longitudinally larger than said means, to allow, through controlled sliding, post-installation compression of said bones.

3. The orthopedic plate system according to claim 1, wherein at least one of said openings is smaller in area at its exit end than its remaining portion in order to firmly control the amount of the motion allowed.

4. The orthopedic plate system according to claim 1, wherein at least one of said screws has a tapered neck.

5. The orthopedic plate system according to claim 1, wherein one or more of said means are bushings.

6. The orthopedic plate system according to claim 5, wherein at least one of said bushings is oval in shape to allow the motion to be greater in one plane and smaller in another, perpendicular plane.

7. The orthopedic plate system according to claim 5, wherein at least two of the exterior and opposite surfaces of said bushings are round.

8. The orthopedic plate system according to claim 1, wherein said motion is at least one member of the group consisting of compression, torsion, flexing, distraction, and bending.

9. A screw and bushing assembly, for securing a bone to an orthopedic plate, which is pre-engineered to allow controlled motion of said bone after the securing of said bone to said plate.

10. The screw and bushing assembly according to claim 9, wherein said bushing is oval in shape to allow more motion in one plane and less motion in another, perpendicular plane.

11. The screw and bushing assembly according to claim 9, wherein said screw has a tapered neck.

12. A method for the installation of an orthopedic plate comprising, in any order, the steps of:
placing said plate, having one or more openings, in the proximity of one or more bones;
screwing one or more screws through said openings into the bone; and,
for one or more of said screws, using means for flexible locking between said plate and the screw, so that controlled motion of the bone is allowed after the installation.

13. The method according to claim 12, wherein at least one of said openings is longitudinally larger than said means, to allow, through controlled sliding, post-installation compression of said bone.

14. The method according to claim 12, wherein at least one of said openings is smaller in area at its exit end than its remaining portion in order to firmly control the amount of the motion allowed.

15. The method according to claim 12, wherein at least one of said screws has a tapered neck.

16. The method according to claim 12, wherein one or more of said means are bushings.

17. The method according to claim 16, wherein at least one of said bushings is oval in shape to allow the motion in one direction and inhibit the motion in another, perpendicular, direction.

18. The method according to claim 16, wherein at least two of the exterior and opposite surfaces of said bushings are round.

19. The method according to claim 12, wherein said motion is at least one member of the group consisting of compression, flexing, distraction and bending.

20. A flexible bushing configured to be positioned between screws and an orthopedic trauma plate, so that the resulting construct is capable of intermittent deformation, which allows intermittent loading of the bone fracture secured by said construct, and the returning of the fracture to at rest state once the deforming force has been released.

21. The flexible bushing according to claim 20, which is ovally shaped to allow the loading to be greater in one plane and smaller in another, perpendicular plane.
